(19) United States
(12) Patent Application Publication
(10) Pub. No.: US 2024/0041306 A1
(43) Pub. Date: Feb. 8, 2024

(54) ENDOSCOPIC FLUID ASPIRATION DEVICE

(71) Applicant: CEDARS-SINAI MEDICAL CENTER, Los Angeles, CA (US)

(72) Inventors: Ali Rezaie, Beverly Hills, CA (US); Mark Pimentel, Los Angeles, CA (US); Srinivas Gaddam, Los Angeles, CA (US); Simon Lo, Rolling Hills Estates, CA (US); Laith Jamil, Bloomfield Hills, MI (US); Kapil Gupta, Santa Monica, CA (US)

(73) Assignee: CEDARS-SINAI MEDICAL CENTER, Los Angeles, CA (US)

(21) Appl. No.: 18/235,945

(22) Filed: Aug. 21, 2023

Related U.S. Application Data
(63) Continuation of application No. 16/489,388, filed on Aug. 28, 2019, now abandoned.
(60) Provisional application No. 62/464,833, filed on Feb. 28, 2017.

(51) Int. Cl. A61B 1/018; A61B 1/00; A61B 1/273; A61B 10/00; A61B 17/34; A61M 1/00

(52) U.S. Cl. CPC A61B 1/018; A61B 1/00016; A61B 1/00082; A61B 1/00085; A61B 1/00094; A61B 1/00135; A61B 1/00137; A61B 1/2736; A61B 10/0045; A61B 17/3478; A61M 1/71; A61M 1/84; A61B 2010/0061; A61B 10/04

(57) ABSTRACT

An endoscopic device for use in a patient's body in disclosed. The endoscopic device may include an outer sheath having a proximal end and a distal end, a suction tube inside the outer sheath, a handle coupled to the distal end of the outer sheath, and a cap on the proximal end of the outer sheath, positioned to seal the proximal end of the outer sheath, wherein the outer sheath comprises a sterile appliance, wherein the cap is structured to open in response to a force applied through the suction tube, and wherein the suction tube comprises an internal cavity.

### CROSS REFERENCE TO RELATED APPLICATION

This application is a continuation of U.S. patent application Ser. No. 16/489,388, filed Aug. 28, 2019, which is the national phase of International Application No. PCT/US2018/019240, filed Feb. 22, 2018 which designated the U.S. and that International Application was published under PCT Article 21(2) in English. This application also claims priority to U.S. Provisional Patent Application No. 62/464,833, filed Feb. 28, 2017, the contents of which are incorporated herein by reference in their entirety.

### FIELD OF THE INVENTION

The present disclosure generally relates to a device and method for aspiration tools including but not limited to a catheter used in endoscopic intervention. More specifically, current device and method relates to a novel catheter that can aspirate small amount of liquid in an uncontaminated fashion with an outer sheath.

### BACKGROUND OF THE DISCLOSURE

Current procedures for collecting of fluid aspirations involve positioning an endoscope in a hollow organ such as small bowel. A sterile catheter is then advanced through the working port of the endoscope, and suction is applied to collect approximately 2 ml of intestinal fluid.

The aspirate is then transferred immediately to aerobic and/or anaerobic sterile tubes for microbiologic analysis.

### SUMMARY OF THE DISCLOSURE

However, there are two key challenges associated with this workflow, specifically: (1) inadequate fluid collection—the ability to collect more than 2 ml of luminal fluid contents within the small intestine is often challenging due to fluid availability; and (2) contamination—liquid is transferred to the catheter from the oral cavity, esophagus, and stomach. For example, bacterial counts in the oral cavity are approximately 10\hat{ }9 CFU/ml, and intestinal aspirates are typically less than 10\hat{ }3 CFU/ml, a contamination rate of just 0.001% could potentially result in a misdiagnosis of Small Intestinal Bacterial or Fungal Overgrowth. Current workflows attempt to address this through sample collection and/or flushing techniques, but there still exists an inherent problem with the device design is present.

As such, obtaining a true sterile sample via an endoscopic device is currently extremely difficult, if not impossible. The endoscopes and their suction channels are constantly being contaminated throughout the procedure. For example, by the time a gastroscope reaches the duodenum, the endoscope is contaminated by the oral, nasopharyngeal, esophageal and gastric microflora.

Therefore, there is a need for device and method for efficient collection of liquid in vivo in a sterile environment.

The following embodiments and aspects thereof are described and illustrated in conjunction with systems, compositions and methods that are meant to be exemplary and illustrative, not limiting in scope.

In an aspect of the present disclosure, an endoscopic device is disclosed. The endoscopic disclosure includes an outer sheath having a proximal end and a distal end, the distal end coupled to a handle; a suction tube inside the outer sheath; and a cap on the proximal end of the outer sheath, positioned to seal the proximal end of the outer sheath, wherein the outer sheath includes a sterile appliance wherein the cap is structured to open responsive to a force applied through the suction tube. The cap may include a plastic, a glucose, or any other material that is nontoxic (or dissolvable) in the body. The force may include air pressure, water pressure, sterile gases, or a force exerted by a guidewire that is configured to deploy through middle of the suction tube. The suction tube may include at least one hole for carrying out aspiration or suction at a target site in vivo. The outer sheath may include a metal coil.

In an embodiment of the present disclosure, the endoscopic device may further include a guidewire that is configured to go through an internal cavity of the suction tube; and an inflatable balloon that is stored near the proximal end of the outer sheath and is configured to deploy outside the outer sheath by a force exerted by the guidewire that is configured to deploy through middle of the suction tube.

In an embodiment of the present disclosure, the endoscopic device may further include a guidewire that is configured to go through an internal cavity of the suction tube; and an elliptical basket that is stored near the proximal end of the outer sheath and is configured to deploy outside the outer sheath by a force exerted by the guidewire that is configured to deploy through middle of the suction tube. The handle may be configured to be connected to a syringe full of an inert gas in order to remove the cap through gas pressure.

In an embodiment of the present disclosure, the endoscopic device may further include a suction tube having a proximal end and a distal end, the distal end coupled to a handle, wherein the suction tube comprises at least one capillary tube.

In an aspect of the present disclosure, a method for using the endoscopic device is disclosed. The method includes: inserting the endoscopic device into a body cavity during an endoscopic surgical procedure; removing the cap; deploying the suction tube; and carrying out aspiration via the suction tube in order to collect a sample from a patient's body.

In another aspect of the present disclosure, a computer readable storage medium tangibly embodying a computer readable program code having computer readable instructions is disclosed. The computer readable instructions, when implemented, cause a computer to carry out the steps of a method as described herein.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 illustrates an example of a system for an endoscopic aspiration that is constructed in accordance with the principles of this disclosure.

FIG. 2 illustrates an example of an endoscopic device that is constructed in accordance with the principles of this disclosure. The cap is expelled or alternatively opened while joined by a hinge.

FIG. 3 illustrates an example of an endoscopic device that is constructed in accordance with the principles of this disclosure. The suction tube is advanced from the outer sheath in order to collect samples.

FIG. 4 illustrates another example of an endoscopic device with a balloon that is constructed in accordance with the principles of this disclosure. The balloon is deflated and stored within the endoscopic device.

FIG. 5 illustrates another example of an endoscopic device with elliptical basket that is constructed in accordance with the principles of this disclosure.

FIG. 6 illustrates, in accordance with various embodiments of the present invention, further views of the non-limiting example shown in FIG. 4. The balloon is inflated with air and deployed outside the endoscopic device within the patient's body.

FIG. 7 illustrates yet another example of an endoscopic device with elliptical basket that is constructed in accordance with the principles of this disclosure.

FIG. 8 illustrates an example of an endoscopic device with capillaries at its end that is constructed in accordance with the principles of this disclosure.

FIG. 9 illustrates an example of an endoscopic device with a moisture absorbing pad.

FIG. 10 illustrates another example of an endoscopic device where a cap is expelled by a guidewire in accordance with the principles of the disclosure.

FIG. 11 illustrates another example of an endoscopic device where a guidewire is removed from the endoscopic device in accordance with the principles of the disclosure.

FIG. 12 illustrates an example of an endoscopic device with a hollow needle knife that is constructed in accordance with the principles of the present disclosure.

### DETAILED DESCRIPTION OF THE DISCLOSURE

The disclosure and the various features and advantageous details thereof are explained more fully with reference to the non-limiting implementations and examples that are described and/or illustrated in the accompanying drawings, and detailed in the following description. It should be noted that the features illustrated in the drawings are not necessarily drawn to scale, and features of one implementation may be employed with other implementations as any person skilled in the art would recognize, even if not explicitly stated herein. Descriptions of well-known components and processing techniques may be omitted so as to not unnecessarily obscure the implementations of the disclosure. The examples used herein are intended merely to facilitate an understanding of ways in which the disclosure may be practiced and to further enable those of skill in the art to practice the implementations of the disclosure. Accordingly, the examples and implementations herein should not be construed as limiting the scope of the disclosure.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.” Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

The terms “patient” and “subject” are used interchangeably herein. These terms are intended to include all animal subjects, including mammals. Human patients/subjects are intended to be within the scope of the patients/subjects treated using the various embodiments of the inventive systems, apparatuses and methods described herein.

A term “wireless transmitter,” as used in this disclosure, means at least one of microwave, Infrared or RF module or a cellular/wireless modem and is configured to transmit data.

The term “coupled” means at least either a direct electrical connection between the connected items or an indirect connection through one or more passive or active intermediary devices. The term “circuit” means at least either a single component or a multiplicity of components, either active and/or passive, that are coupled together to provide a desired function. The term “signal” as used herein may include any meanings as may be understood by those of ordinary skill in the art, including at least an electric or magnetic representation of current, voltage, charge, temperature, data or a state of one or more memory locations as expressed on one or more transmission mediums, and generally capable of being transmitted, received, stored, compared, combined or otherwise manipulated in any equivalent manner.

Terms such as “providing,” “processing,” “supplying,” “determining,” “calculating” or the like may refer at least to an action of a computer system, computer program, signal processor, logic or alternative analog or digital electronic device that may be transformative of signals represented as physical quantities, whether automatically or manually initiated.

A “computer,” as used in this disclosure, means any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a cloud, a super computer, a personal computer, a laptop computer, a palmtop computer, a mobile device, a tablet computer, a notebook computer, a desktop computer, a workstation computer, a server, or the like, or an array of processors, microprocessors, central processing units, general purpose computers, super computers, personal computers, laptop computers, palmtop computers, mobile devices, tablet computers, notebook computers, desktop computers, workstation computers, servers, or the like.

A “server,” as used in this disclosure, means any combination of software and/or hardware, including at least one application and/or at least one computer to perform services for connected clients as part of a client-server architecture. The at least one server application may include, but is not limited to, for example, an application program that can accept connections to service requests from clients by sending back responses to the clients. The server may be configured to run the at least one application, often under heavy workloads, unattended, for extended periods of time with minimal human direction. The server may include a plurality of computers configured, with the at least one application being divided among the computers depending upon the workload. For example, under light loading, the at least one application can run on a single computer. However, under heavy loading, multiple computers may be required to run the at least one application. The server, or any if its computers, may also be used as a workstation.

A “database,” as used in this disclosure, means any combination of software and/or hardware, including at least one application and/or at least one computer. The database may include a structured collection of records or data organized according to a database model, such as, for example, but not limited to at least one of a relational model, a hierarchical model, a network model or the like. The database may include a database management system application (DBMS) as is known in the art. The at least one application may include, but is not limited to, for example, an application program that can accept connections to service requests from clients by sending back responses to the clients. The database may be configured to run the at least one application, often under heavy workloads, unattended, for extended periods of time with minimal human direction.

A “communications network,” as used in this disclosure, means a wired and/or wireless medium that conveys data or information between at least two points. The wired or wireless medium may include, for example, a metallic conductor link, a radio frequency (RF) communication link, an Infrared (IR) communication link, telecommunications networks, an optical communication link, internet (wireless and wired) or the like, without limitation. The RF communication link may include, for example, WiFi, WiMAX, IEEE 802.11, DECT, 0G, 1G, 2G, 3G, 4G, 5G or future cellular standards, Bluetooth, Bluetooth Low Energy, NFC, ultrasound, induction, laser (or similar optical transmission) and the like.

A “computer-readable storage medium,” as used in this disclosure, means any medium that participates in providing data (for example, instructions) which may be read by a computer. Such a medium may take many forms, including non-volatile media, volatile media, and transmission media. Non-volatile media may include, for example, optical or magnetic disks, flash memory, and other persistent memory. Volatile media may include dynamic random access memory (DRAM). Transmission media may include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read. The computer-readable medium may include a “Cloud,” which includes a distribution of files across multiple (e.g., thousands of) memory caches on multiple (e.g., thousands of) computers.

Various forms of computer readable media may be involved in carrying sequences of instructions to a computer. For example, sequences of instruction (i) may be delivered from a RAM to a processor, (ii) may be carried over a wireless transmission medium, and/or (iii) may be formatted according to numerous formats, standards or protocols, including, for example, WiFi, WiMAX, IEEE 802.11, DECT, 0G, 1G, 2G, 3G or 4G cellular standards, Bluetooth, or the like.

A “network,” as used in this disclosure means, but is not limited to, for example, at least one of a local area network (LAN), a wide area network (WAN), a metropolitan area network (MAN), a personal area network (PAN), a campus area network, a corporate area network, a global area network (GAN), a broadband area network (BAN), a cellular network, the Internet, the cloud network, or the like, or any combination of the foregoing, any of which may be configured to communicate data via a wireless and/or a wired communication medium. These networks may run a variety of protocols not limited to TCP/IP, IRC, SSL, TLS, UDP, or HTTP.

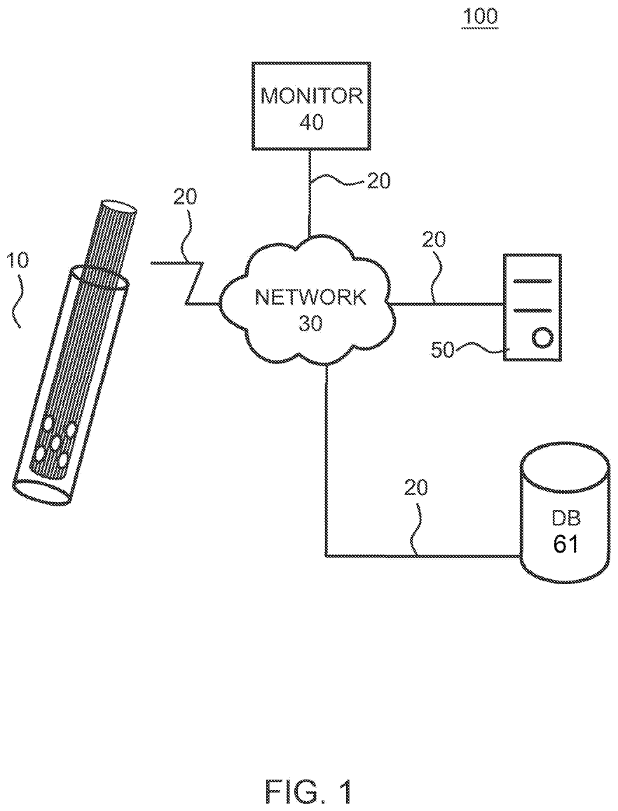

FIG. 1 shows an example of a system that is constructed according to the principles of the disclosure that provides wireless navigation of presently disclosed endoscopic device to a treatment (or suction) site of a patient, and receiving and carrying out wireless transmission of instructions, such as, for example, begin aspiration, deploy balloon, remove cap, apply treatment, and the like. The system 100 includes at least one endoscopic device (or aspiration catheter) 10, a network 30, a monitor (e.g., a system manager) computer (or computing device) 40, a hosted server (or computer) 50, and a database 61, all of which may be coupled to each other via communication links 20. For instance, the hosted server 50 and database 61 may be connected to each other and/or the network 30 via one or more communication links 20. The at least one endoscopic device 10 and the monitor computer 40 may be coupled to the network 30 via communication links 20. The at least one endoscopic device 10 may be used by, for example, an authorized user (e.g., doctor, nurse, or the like) of a patient to whom the at least one endoscopic device 10 is being used. Once the at least one endoscopic device 10 collects liquid in vivo, said liquid may then transferred immediately to aerobic and/or anaerobic sterile tubes for microbiologic analysis.

The at least one endoscopic device 10, the monitor computer 40, the hosted server 50, and the database 61 may each include a computer-readable medium including a computer program that may be executed to carry out the processes disclosed herein. The computer-readable medium may include a code section or code segment for performing each step disclosed herein.

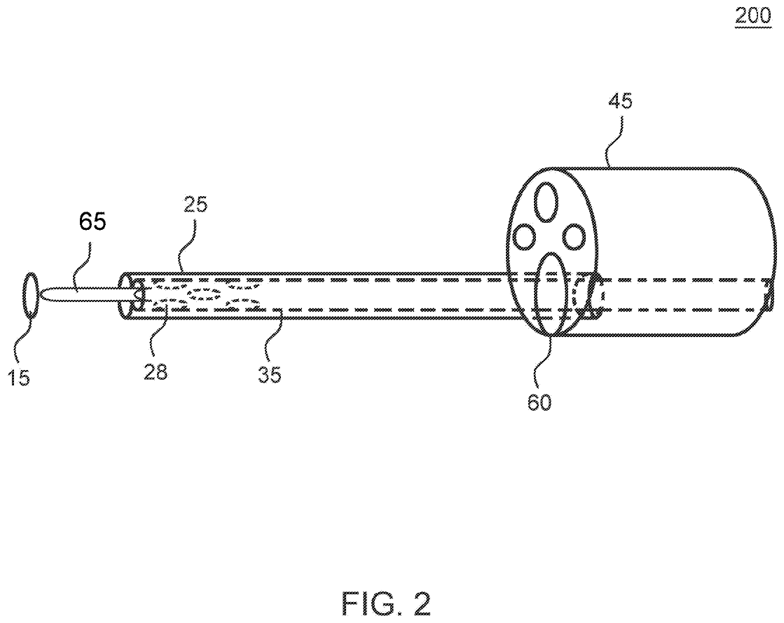

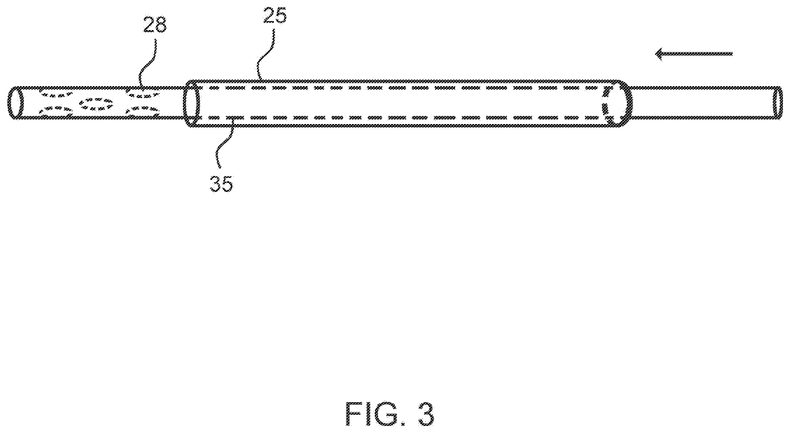

FIG. 2 illustrates an example of an endoscopic device (or aspiration catheter) 200 that is constructed in accordance with the principles of this disclosure. The aspiration catheter 200 includes an outer, sealed sheath (e.g., outer metal coil) 25 for maintaining a sterile environment for the suction tube 35 inside the sheath 25 until the endoscopic device 200 is navigated to the aspiration site in the patient's body (e.g., distal part of the duodenum, proximal portion of the jejunum, or the like). In some examples, the outer sheath 25 may include a cap 15 that seals the suction tube 35 inside of the sheath 25. After navigating the endoscope 45 to the aspiration site, the cap 15 may be expelled to reveal the sterile suction tube 35. Alternatively, the cap 15 can be opened while joined by a hinge (not shown) to tip of the endoscopic device 200. The cap 15 may be dislodged by pushing, air, water, sterile gases (e.g., CO₂), or a guidewire 65 that is configured to navigate through the middle of the suction tube 35 to expel the cap 15. At this point (or concurrently), the sterile suction tube 35 may then be pushed out of the outer sheath 25 in order to conduct aspiration (or suction) in vivo (as shown in, e.g., FIG. 3). Alternatively, the guidewire 65 may be first removed before the sterile suction tube 35 is pushed out of the outer sheath 25 to conduct aspiration. The suction tube 35 may include at least one hole 28 for carrying out aspiration/suction. The cap 15 may include a plastic, penetrable membrane, or made from a dissolvable material (e.g., wax or glucose crystal for bronchoscopy or cystoscopy) so that it may harmlessly dissolve in the body. The cap 15 may also be made from any other material that may be easily dissolved in a person's body or be naturally excreted from the body. Additionally, the cap 15 may also include any non-toxic material.

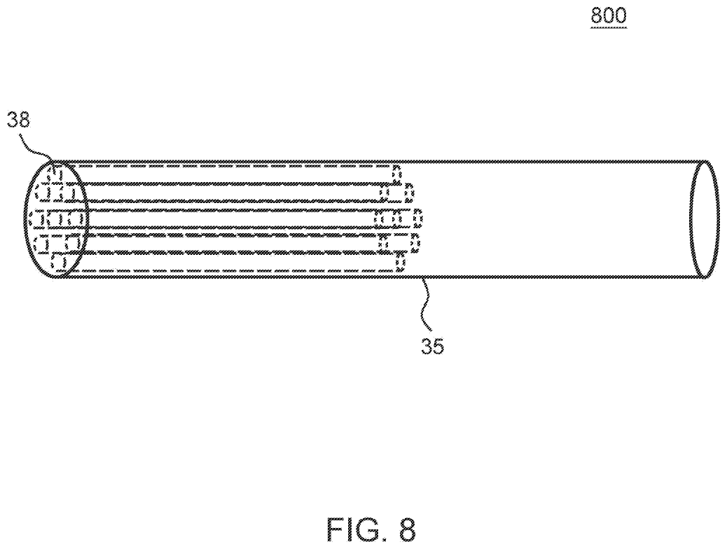

Alternatively (or additionally) and referring to FIGS. 2 and 8 concurrently, the suction tube 35 may include at least one capillary tube 38 (as shown in, e.g., FIG. 8) and may be configured to pass through a hole 60 on an endoscope 45 with or without a covering sheath 25 and enable the aspiration catheter 200 to aspirate even very small amounts (e.g., less than 2 ml) of liquid from the target organ (e.g. small bowel, biliary tree, gallbladder, bladder, intraabdominal/thoracic cavity, sinuses or lung). This addresses deficiencies in currently available technologies in that simple porous catheters are not able to suction small amount of fluids or do it without contamination. By incorporating capillary tubes 38, the disclosed endoscopic device (or aspiration catheter) 200 provides medical professionals with an ability to suction even small amount of liquid. Furthermore, the capillary tube design may be configured to prevent the suctioned fluid from refluxing back and allow the device to aspirate in an uncontaminated fashion with or without an outer sheath.

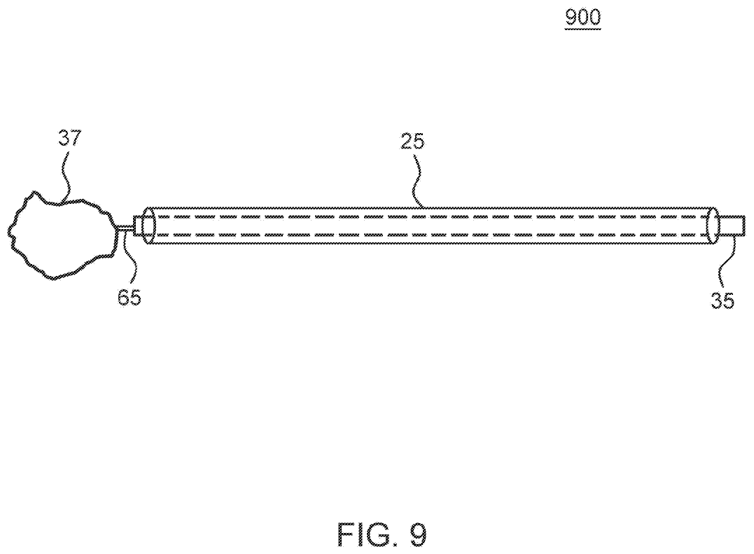

Additionally, as shown in, e.g., FIG. 9, the endoscopic device 900 may include a moisture absorbing pad 37 attached to the guidewire 65 that may be deployed (or continue to be deployed during the in vivo procedure) so that the pad 37 can absorb liquid and keep the endoscopic device 900 sterile during insertion in vivo. The moisture absorbing pad 37 may be configured to be stored inside the suction tube 35 during insertion in vivo and be deployed after the endoscopic device 900 reaches a target site inside the patient's body.

Referring to FIGS. 1-6 concurrently, the presently disclosed endoscopic device, method, and system allow the endoscopic device 200 to maintain an aspiration/suction tube 35 that is not contaminated with bacteria from the body prior to arriving at the site inside the patient's body and taking a sample of, e.g., liquid, skin, bacteria, and the like. This also allows the aspiration catheter to aseptically suction a sample (e.g., from the small bowel) without contaminating it with bacteria from the oral, esophageal, and other passages that are traversed while travelling to the aspiration site.

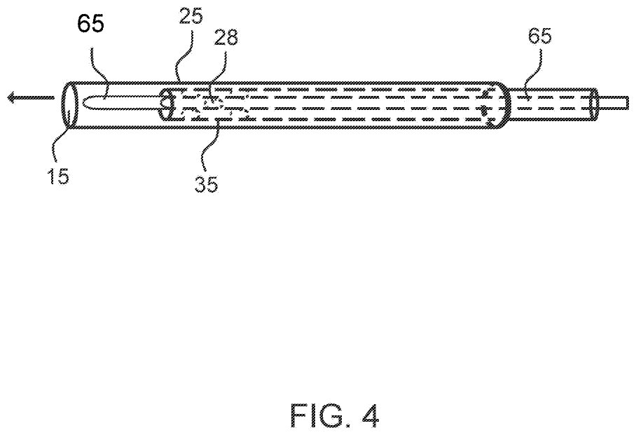

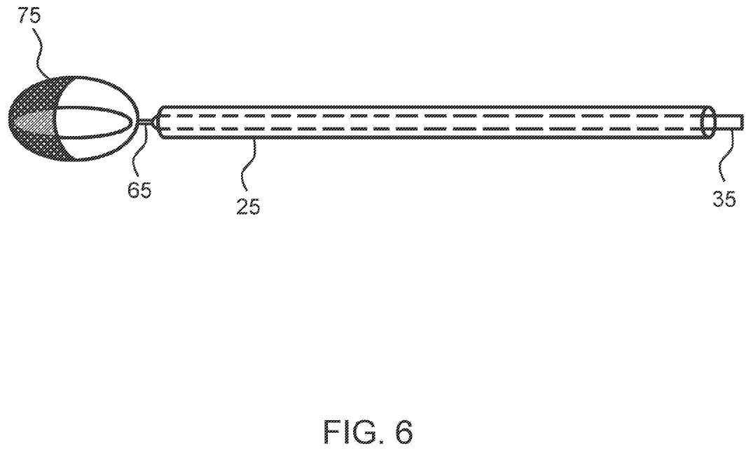

Additionally, as shown in FIG. 4, the guidewire 65 may be attached to an inflatable balloon 75 that inflates distal to the suction tube 35 in order to plug the lumen during suction. After the insertion, the cap 15 may be removed and the balloon 75 may be deployed and inflated as shown in, e.g., FIG. 6. As shown in FIG. 6, the balloon 75 is inflated, the suction tube 35 (or the catheter) may be withdrawn in order to pool more luminal contents 58 and such contents 58 are suctioned by the at least one hole 28.

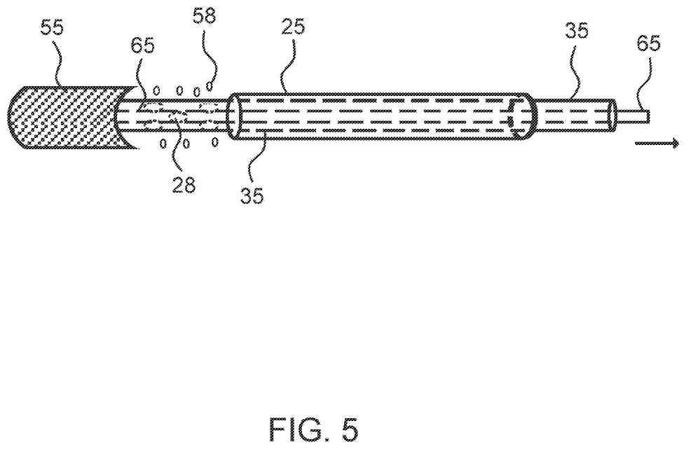

FIG. 5 illustrates an example of the guidewire 65 that may be attached to an elliptical basket 55 that may be deployed once the porous tube is unsealed from the sheath. As shown in FIG. 5, the disclosed endoscopic device (or catheter) passes through the scope and due to its unique multilayer design, it can suction the liquid contents in a completely uncontaminated and even anaerobic fashion.

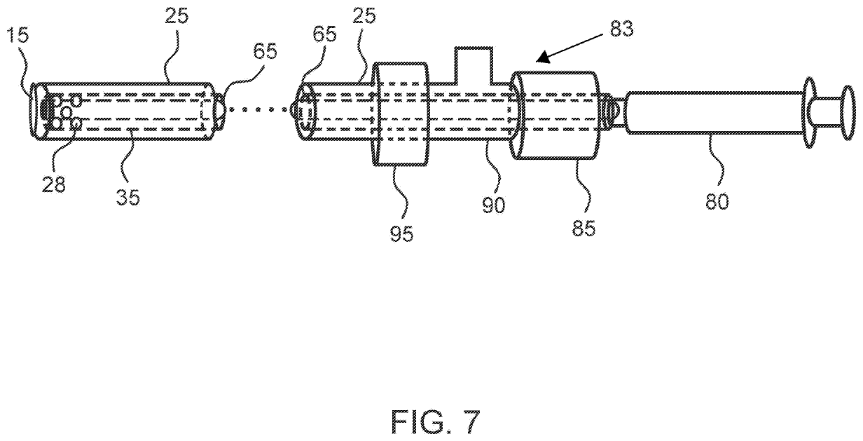

As shown in FIG. 7, the catheter/suction tube may be connected to a syringe 80 full of an inert gas or CO₂ in order to remove the cap 15 through gas pressure. As further illustrated in FIG. 7, a stopper and handle system 83 (e.g., stoppers 85, 90, and 95) may be utilized to manipulate the catheter and extend the suction tube 35. For example, a stopper 85 may include a non-removable plastic stopper sealed to the middle suction tube 35 and may further be configured to connect to a syringe 80. The stopper 85 may also be configured to allow a guidewire to pass through it. In another example, a stopper 90 may include a removable plastic stopper, wherein the width of the stopper 90 determines the length of porous middle tube (e.g., middle suction tube 35) that can be advanced into the lumen in, e.g., catheter 200 in FIG. 2. In yet another example, a stopper 95 may include a non-removable plastic stopper that is sealed to outer coil (e.g., outer sheath 25) but configured so that the middle tube passes through it freely.

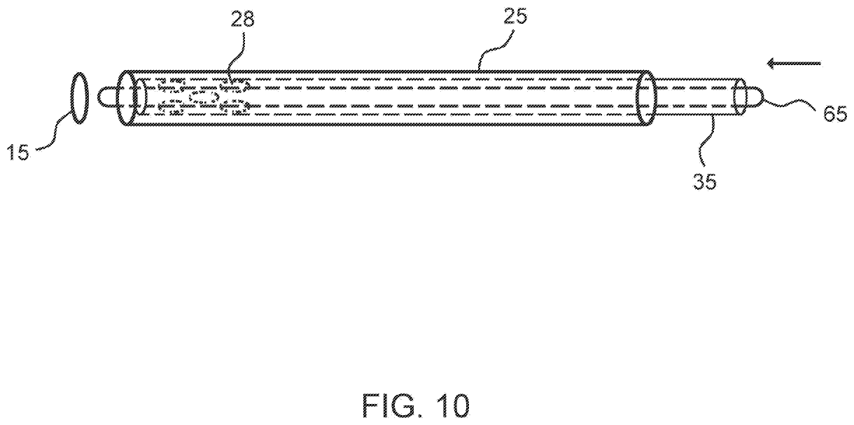

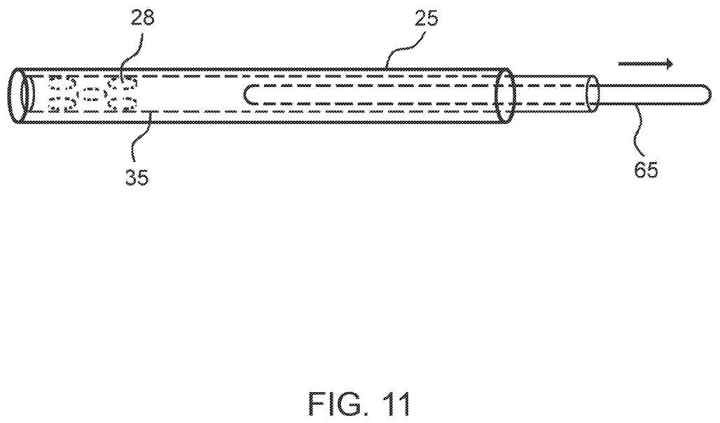

FIGS. 3, 10, and 11 illustrate an example of an endoscopic device being used in accordance with the principles of the present disclosure. Referring to FIGS. 3, 10, and 11 concurrently, the endoscopic device includes a guidewire 65, a suction tube 35 that encapsulates the guidewire 65, an outer sheath 25 with a proximal end that includes a cover and a distal end that is configured to be connected to, e.g., a handle, a syringe, a machine, and the like. As shown in, e.g., FIG. 10, the guidewire 65 is pushed towards the proximal end to separate (expel) the cap 15 from the suction tube 35. Alternatively, the cap 15 may be pulled out of the suction tube along with the guidewire 65 as the guidewire 65 is pulled out of the suction 35 towards the distal end of the outer sheath 25. Once the cap 15 is removed and the guidewire 65 is removed from the suction tube 35 as shown in, e.g., FIG. 11, the suction tube 35 can advance out of the outer sheath 25 and begin aspiration (or suctioning) the luminal contents in sterile environment via a hole 28 on the suction tube 35 (see, e.g., FIG. 3).

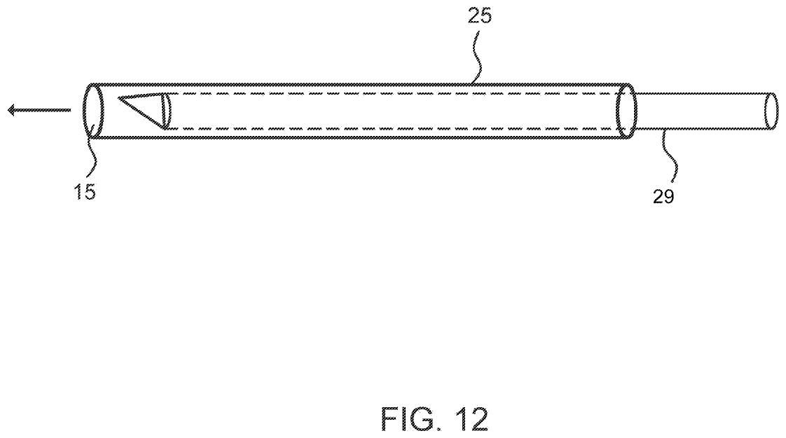

FIG. 12 illustrates an example of an endoscopic device with a hollow needle knife 29 that is constructed in accordance with the principles of the present disclosure. The hollow needle knife 29 may be enclosed in an outer sheath 25, wherein a force (e.g., air, push, hinge mechanism) would open a cap 15 so that the hollow needle knife 29 can be deployed and used for, e.g., surgical procedures. Further, the hollow needle knife 29 may include at least one hole to carry out suctioning of samples in vivo.

As disclosed herein, the present invention provides a unique multilayered tip and a handle design of a catheter that can suction the contents in a completely sterile and even anaerobic fashion. It addresses following problems that are encountered during in vivo endoscopic procedure: (1) inability to confidently identify the pathogens (as oppose to contaminant bacteria, fungus and viruses) in diseases such as small intestinal bacterial/fungal overgrowth, infectious enteritis, infectious cystitis and nephritis, respiratory tract infections (e.g. ventilator associated pneumonia), sinusitis, cholangitis and pancreatitis; (2) inability to accurately identify anaerobic pathogens due to exposure to open air; (3) inability to accurately develop microbiome banks due to poor current techniques; and (4) inability to confirm the eradication of infection. Furthermore, by utilizing capillary tubes, the current invention provides an ability to suction even small amount of liquid in a sterile environment and with (or without) an outer sheath.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. An endoscopic device for use in a patient's body, comprising:
an outer sheath having a proximal end and a distal end;
a suction tube inside the outer sheath and configured to advance out of the outer sheath;
a removable guidewire encapsulated in the suction tube and configured to be pushed towards the proximal end of the outer sheath or pulled out of the suction tube towards the distal
a handle coupled to the distal end of the outer sheath; and
a cap on the proximal end of the outer sheath, positioned to seal the proximal end of the outer sheath,
wherein the suction tube comprises an internal cavity protected by the outer sheath and the cap, and
wherein the cap is structured to open in response to a force generated by the guidewire and applied through the suction tube.

2. The endoscopic device of claim 1, wherein the cap comprises at least one of:
a plastic, a glucose, and any other material that is nontoxic in the body.

3. The endoscopic device of claim 1, wherein the cap comprises a material that is dissolvable in the body.

4. The endoscopic device of claim 1, wherein the force comprises at least air pressure, water pressure, or sterile gases, or any combination of air pressure, water pressure, and/or sterile gases exerted by the guidewire that is configured to deploy through a middle of the suction tube.

5. The endoscopic device of claim 1, wherein the suction tube comprises at least one hole or a plurality of holes for carrying out aspiration at a target site inside the patient's body.

6. The endoscopic device of claim 1, wherein the outer sheath comprises a metal coil.

7. The endoscopic device of claim 1, further comprising:
an inflatable balloon that is stored near the proximal end of the outer sheath and is configured to deploy outside the outer sheath by a force exerted by the guidewire that is configured to deploy through a middle of the suction tube.

8. The endoscopic device of claim 1, further comprising:
an elliptical basket that is stored near the proximal end of the outer sheath and is configured to deploy outside the outer sheath by a force exerted by the guidewire that is configured to deploy through a middle of the suction tube.

9. The endoscopic device of claim 1, wherein the handle is configured to be connected to a syringe filled with gas, the syringe connected to the suction tube in order to remove the cap through gas pressure generated by the syringe.

10. The endoscopic device of claim 9, wherein the gas is an inert gas.

11. A method for using an endoscopic device inside a patient's body comprising:
inserting the endoscopic device into a body cavity during an endoscopic surgical procedure,
wherein the endoscopic device comprises:
an outer sheath having a proximal end and a distal end,
a suction tube inside the outer sheath and configured to advance out of the outer sheath,
a removable guidewire encapsulated in the suction tube and configured to be pushed towards the proximal end of the outer sheath or pulled out of the suction tube towards the distal end of the outer sheath,
a handle coupled to the distal end of the outer sheath, and
a cap on the proximal end of the outer sheath and positioned to seal the proximal end of the outer sheath,
wherein the suction tube comprises an internal cavity protected by the outer sheath and the cap, and
wherein the cap is structured to open in response to a force generated by the guidewire and applied through the suction tube;
removing the cap;
deploying the suction tube; and
carrying out an aspiration via the suction tube in order to collect a sample from the patient's body.

12. The method of claim 11, wherein the cap comprises a material that is non-toxic and dissolvable inside the patient's body.

13. The method of claim 11, wherein the force comprises at least air pressure, water pressure, or sterile gases, or any combination of air pressure, water pressure, and/or sterile gases exerted by the guidewire that is configured to deploy through a middle of the suction tube.

14. The method of claim 11, wherein the suction tube comprises at least one hole or a plurality of holes for carrying out the aspiration inside the patient's body.

15. The method of claim 11, wherein the outer sheath comprises a metal coil.

16. The method of claim 11, wherein the endoscopic device further comprises:
an inflatable balloon that is stored near the proximal end of the outer sheath and is configured to deploy outside the outer sheath by a force exerted by the guidewire that is configured to deploy through a middle of the suction tube, and
wherein the method further comprises inflating the balloon with air and deploying the inflated balloon outside the endoscopic device within the patient's body.

17. The method of claim 11, wherein the endoscopic device further comprises:
an elliptical basket that is stored near the proximal end of the outer sheath and is configured to deploy outside the outer sheath by a force exerted by the guidewire that is configured to deploy through a middle of the suction tube, and
wherein the method further comprises deploying the elliptical basket outside the endoscopic device within the patient's body.

18. The method of claim 11, wherein the handle is configured to be connected to a syringe filled with an inert gas, the syringe connected to the suction tube in order to remove the cap through gas pressure generated by the syringe.

19. A system for carrying out an aspiration inside a patient's body comprising:
an endoscopic device comprising
an outer sheath having a proximal end and a distal end,
a suction tube inside the outer sheath and configured to advance out of the other sheath,
a removable guidewire encapsulated in the suction tube and configured to be pushed towards the proximal end of the outer sheath or pulled out of the suction tube towards the distal end of the outer sheath,
a handle coupled to the distal end of the outer sheath, and
a cap on the proximal end of the outer sheath and positioned to seal the proximal end of the outer sheath,
wherein the suction tube comprises an internal cavity protected by the outer sheath and the cap, and
wherein the cap is structured to open in response to a force generated by the guidewire and applied through the suction tube;
a network;
a monitor computer;
a hosted server; and
a database,
wherein the network, the monitor computer, the hosted server, and the database are all coupled to each other via communication links.

20. The system of claim 19, wherein the endoscopic device, the monitor computer, the hosted server, and the database each comprises a computer-readable medium tangibly embodying a computer readable program code having computer readable instructions which, when implemented, cause the system to carry out the steps of:
removing the cap;
deploying the suction tube; and
carrying out an aspiration via at least one hole or a plurality of holes of the suction tube in order to collect a sample from the patient's body.
